(19) United States
(12) Patent Application Publication
(10) Pub. No.: US 2010/0297026 A1
(43) Pub. Date: Nov. 25, 2010

(54) Imaging of Activated Vascular Endothelium Using Immunomagnetic MRI Contrast Agents

(76) Inventor: Gerald V. Doyle, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/445,652

(22) PCT Filed: Nov. 1, 2007
(86) PCT No.: PCT/US07/23048

§ 371 Date: Apr. 15, 2009

Related U.S. Application Data
(60) Provisional application No. 60/856,127, filed on Nov. 2, 2006.

(51) Int. Cl. A61K 49/14; A61K 49/16

(52) U.S. Cl. 424/9.323

(57) ABSTRACT

Immunomagnetic nanoparticles are used as a contrast agent for enhancing medical diagnostic imaging such as magnetic resonance imaging (MRI). The present invention is directed to methods of making targeted MRI contrast agents from immunomagnetic particles, and to methods of using such MRI contrast agents. Typically, such targeted MRI contrast agents provide enhanced relaxivity, improved signal-to-noise, targeting ability, and resistance to agglomeration. Methods of making such MRI contrast agents typically afford better control over particle size, and methods of using such MRI contrast agents typically can afford enhanced blood clearance rates and distribution. The ability to use the contrast agents im MRI provides a tool in the diagnosis and treatment of several disease states.

### CROSS REFERENCE TO RELATED APPLICATIONS

This application is a non-provisional application, which is incorporated by reference herein and claims priority, in part, of U.S. Provisional Application No. 60/856,127, filed 2 Nov. 2006.

### FIELD OF THE INVENTION

This invention relates generally to in vivo diagnostic imaging with the use of nanoparticles. More specifically, this invention relates to a diagnostic imaging technique in which a disease state may be imaged using a targeted contrast agent formed by functionalizing nanoparticles in a coating process that incorporates a targeted moiety. These contrast agents are suitable for magnetic resonance imaging used to assess, diagnose, and treat disease states such as, but not limited to, cancer, cardiovascular, cerebrovascular, peripheral vascular, auto immune and all inflammatory diseases.

### BACKGROUND OF THE INVENTION

The present invention relates to immunomagnetic nanoparticles as contrast agent and their use in medical diagnostic imaging techniques such as, but not limited to, magnetic resonance imaging (“MRI”). The present invention is based upon the novel ability of these particles to remain suspended and not aggregate, their coating compositions which prevent particle aggregation thereby improving particle stability, their ability to permit functionalization of the particle surface, and methods for their efficient manufacture.

The use of contrast agents in diagnostic medicine is rapidly growing. In X-ray diagnostics, for example, increased contrast of internal organs, such as the kidneys, the urinary tract, the digestive tract, the vascular system of the heart (angiography), etc., is obtained by administering a contrast agent which is more radiopaque than the surrounding tissue, organ or spaces. In ultrasound diagnostics, improved contrast is obtained by administering compositions having acoustic impedances different than that of blood and other tissues.

In proton MRI diagnostics, increased contrast of internal organs and tissues may be obtained by administering compositions containing paramagnetic metal species. For example, hydroxylapatite particles are used for enhancing medical imaging of body organs and tissues. These particles are composed of the mineral calcium apatite with the formula Ca₅(PO₄)₃(OH). It is the inorganic mineral component of bone and teeth. Because of its paramagnetic metal ions, it is useful in magnetic resonance imaging, X-ray or ultrasound imaging of liver and spleen (U.S. Pat. No. 5,690,908).

In general for contrast agents to be effective, they must interfere with the wavelength of electromagnetic radiation used in the imaging technique, alter the physical properties of tissue to yield an altered signal, or provide the source of radiation itself. Commonly used materials include organic molecules, metal ions, salts or chelates, particles (particularly iron particles), or labeled peptides, proteins, polymers or liposomes. After administration, the agent may non-specifically diffuse throughout body compartments prior to being metabolized and/or excreted; these agents are generally known as non-specific agents. Alternatively, the agent may have a specific affinity for a particular body compartment, cell, organ, or tissue; these agents can be referred to as targeted agents.

For agents injected or absorbed into the body and distributed by the blood, it is desirable to have an appropriate blood half-life (U.S. Pat. No. 7,229,606). While extremely long half-lives (i.e., days or weeks) are unnecessary in clinical imaging situations and possibly dangerous (due to the increased chance for toxicity and metabolic breakdown into more toxic molecules), short half-lives are also not desirable. If the image enhancement lasts for too short of time, it is difficult to acquire a high-quality image of the patient. In addition, rapid clearance of a targeted agent will reduce the amount of the agent available to bind to the target site and thus reduce the “brightness” of the target site on the image.

Magnetic resonance imaging (MRI) is a technique that uses a powerful magnetic field and radio signals to create sophisticated vertical, cross-sectional and three-dimensional images of structures and organs inside a body. MRI is most effective at providing images of tissues and organs that contain water, such as the brain, internal organs, glands, blood vessels and joints. When focused radio wave pulses are broadcast towards magnetically aligned hydrogen atoms in a tissue of interest, the hydrogen atoms return a signal as a result of proton relaxation. The subtle differences in the signal from various body tissues enable MRI to differentiate organs, and potentially contrast benign and malignant tissue, making MRI useful for detecting tumors, bleeding, aneurysms, lesions, blockage, infection, joint injuries, etc.

When used in MRI, contrast agents change the relaxation time of the tissues they occupy. Contrast agents for MRI are typically magnetic materials that enhance the relaxation time of the water protons in a close range due to a time-dependent magnetic dipolar interaction between the magnetic moments of the contrast agent and the water protons. MRI contrast agents are either positive agents that brighten the tissue that they occupy, or they are negative agents that make a tissue appear darker. For in vivo diagnostics, MRI provides good resolution characteristics (ca. 2 mm), however, it offers poor sensitivity when compared with other imaging techniques. The administration of contrast agents greatly improves imaging sensitivity. Paramagnetic gadolinium (Gd) species such as Gd-DTPA (e.g., OMNISCAN®) brighten the tissue and have been used clinically as contrast agents in MRI.

Contrast agent specificity is a desired property for enhancing signal-to-noise ratio at a site of interest and providing functional information through imaging. Natural distribution of contrast agents depends upon the size, charge, surface chemistry and administration route. Contrast agents may concentrate at healthy tissue or lesion sites and increase the contrast between the normal tissue and the lesion. In order to increase contrast, it is necessary to concentrate the agents at the site of interest and increase relaxivity. In addition, it is also desirable to increase the uptake of the agents by diseased cells in relation to healthy cells.

Most contrast agents are somewhat organ-specific due to the fact that they are excreted either by the liver or by the kidneys. Initial studies using gadolinium chelates as receptor-directed agents required a high level of contrast agent for a significantly reduced relaxation (Eur. Radiol. 2001. 11:2319-2331, Y.-X. J. Wang, S. M. Hussain, G. P. Krestin). Compared to the gadolinium chelates, magnetite particles possess about two to three orders of magnitude greater magnetic susceptibility (Eur. Radiol. 2001. 11:2319-2331, Y.-X. J. Wang, S. M. Hussain, G. P. Krestin). Therefore, iron oxide contrast agents potentially offer a stronger signal at a lower dosage than gadolinium chelates. The higher sensitivity of iron oxide agents provides additional benefits due to the limited number of targets available to bind with in a given tissue.

There are a variety of magnetic nanoparticles such as magnetodedrimers, magnetoliposomes and polymer-coated nanoparticles (such as dextran, polyvinyl alcohol, etc.) that are made up of crystalline superparamagnetic iron oxide nanoparticles embedded in an organic coating.

Most of the commercial contrast agents are based on dextran or dextran derivatives, where relatively small size particles are employed. However, dextran coatings have been claimed to be unstable at the alkaline conditions of the particle synthesis, and their chemical composition has therefore been questioned. Additionally, dextran-induced anaphylactic reactions present potential problems (U.S. Pat. No. 5,492,814).

Conventionally, iron oxide nanoparticles are synthesized and precipitated from alkaline aqueous solutions in the presence of water soluble organic molecules such as dextran, and such nanoparticles generally have an organic coating. Nanoparticles obtained by such methods tend to have a broad size distribution of the paramagnetic iron oxide, and, as a result, the coated particles also exhibit a broad size distribution. In addition, this method provides little control over the degree of coating leading to particles containing multiple iron oxide nanoparticles within a single agent. Extensive manufacturing techniques, including multiple purification and size separation steps, are necessary to obtain the desired particle sizes. Particle size, as well as the organic coating composition, is very important as it directly affects the pharmacokinetics of the nanoparticles. The size of the iron oxide directly relates to the paramagnetism and the relaxivity of the agent. Therefore, a broad size distribution generally translates into an average sensitivity.

Nanoparticles obtained using conventional methods also have a low level of crystallinity, which significantly impacts the sensitivity of the contrast agent. Moreover, nanoparticles tend to agglomerate due to their high surface energy, which is a significant problem encountered during synthesis and purification steps. Such agglomeration increases the size of the particle, resulting in rapid blood clearance as well as reducing targeting efficiency, and may result in a reduction in relaxivity. Size, blood circulation time and the organic coating affect the targeting efficiency in different ways. When large particles are employed, only a few targeting ligands may be attached before the particles become large enough to be cleared from the blood and failure of the agent to reach the intended target. Smaller particle sizes may be much “stickier” at the sites where the recognition between the biomarker and the ligand occurs. When coatings are globular, reactive sites intended for ligand attachment are generally hindered, thereby reducing conjugation efficiency. In addition, once bound, ligands may reside in the interior of globular coatings, preventing easy access to the biomarkers.

Current imaging agents and their use primarily provide anatomical information. However, underlying disease states are biochemical processes that propagate the disease well before outward physical symptoms appear. Having the ability to image the biochemical pathways, or specific markers in the pathways, in the early stages of the disease would provide functional information.

Contrast agents that are targeted towards particular molecular markers that are able to detect the increased presence of the crucial chemical biomarkers, and thereby provide biochemical information on the early presence of a specific disease state, are needed. Molecular contrast agents capable of targeting sites of a lesion are needed to address the medical need for early diagnosis and treatment of disease. One of the major developmental needs in molecular imaging and targeted delivery of contrast agents is the identification of the biomarkers. Contrast agents, however, have inherent problems that limit targeting efficiency, such as low sensitivity, low signal-to-noise ratio, large particle sizes, rapid blood clearance, low efficiency of ligand attachment and the accessibility of ligands to the biomarkers' targets.

Previous examples of targeted delivery of contrast agents involved using iron oxide nanoparticles coated with cross-linked dextran and subsequently adding antibodies or peptides (Kelly, K. A., Allport, J. R., Tsourkas, A., Shinde-Patil, V. R., Josephson, L., and Weissleder, R. (2005) Circ Res 96, 327-336; Wunderbaldinger, P., Josephson, L., and Weissleder, R. (2002) Bioconjug Chem 13, 264-268). While conjugation of the molecules and delivery of agent to a site of interest was accomplished, the agents became very large (>65 nm) upon bioconjugation and demonstrated very low blood half-life (<50 minutes) which could dramatically affect efficacy in humans.

A few paramagnetic iron oxide nanoparticles that have been evaluated in medicine as MRI contrast agents. Some of these products are available on the market, such as Feridex IV®, Abdoscan® and Lumirem® as contrast agents used in clinical applications for liver and spleen imaging. Nanoparticles are classified on the basis of size, large (1.5 to about 50 microns) small (0.7-1.5 microns) or colloidal (<200 nm). The latter, which are also known as ferrofluids or ferrofluid-like materials, are sometimes referred to herein as colloidal, paramagnetic particles.

Small magnetic particles of the type described above have been shown to be quite useful in analyses involving bio-specific affinity reactions, as they are conveniently coated with biofunctional polymers (e.g., proteins), provide very high surface areas and give reasonable reaction kinetics. Magnetic particles ranging from 0.7-1.5 microns have been described in the patent literature, including, by way of example, U.S. Pat. Nos. 3,970,518; 4,018,886; 4,230,685; 4,267,234; 4,452,773; 4,554,088; and 4,659,678.

Small magnetic particles, such as those mentioned above, generally fall into two broad categories. The first category includes particles that are permanently magnetizable, or ferromagnetic; and the second comprises particles that exhibit bulk magnetic behavior only when subjected to a magnetic field. The latter are referred to as magnetically responsive particles. Materials displaying magnetically responsive behavior are sometimes described as paramagnetic. However, materials normally considered ferromagnetic, e.g., magnetic iron oxide, may be characterized as paramagnetic when provided in crystals of about 30 nm or less in diameter. Relatively larger crystals of ferromagnetic materials, by contrast, retain permanent magnet characteristics after exposure to a magnetic field and tend to aggregate thereafter due to strong particle-particle interactions. Like the small magnetic particles mentioned above, large magnetic particles (>1.5 microns to about 50 microns) can also exhibit paramagnetic behavior. Typical of such materials are those described by Ugelstad in U.S. Pat. No. 4,654,267 and manufactured by Dynal, (Oslo, Norway).

U.S. Pat. No. 4,795,698 to Owen et al. relates to polymer-coated, colloidal, paramagnetic particles which are produced by the formation of magnetite from Fe⁺²/Fe⁺³ salts in the presence of polymer. U.S. Pat. No. 4,452,773 to Molday describes a material similar in properties to those described in Owen et al., which is produced by forming magnetite and other iron oxides from Fe⁺²/Fe⁺³ via base addition in the presence of very high concentrations of dextran. The resulting particles from both procedures exhibit an appreciable tendency not to settle from aqueous suspensions for observation periods as long as several months. Materials so produced have colloidal properties and have proved to be very useful in cell separation. The Molday technology has been commercialized by Miltenyi Biotec, Bergisch Gladbach, Germany and Terry Thomas, Vancouver, Canada.

Another method for producing paramagnetic, colloidal particles is described in U.S. Pat. No. 5,597,531. In contrast to the particles described in the Owen et al., or Molday patents, these latter particles are produced by directly coating a biofunctional polymer onto pre-formed superparamagnetic crystals which have been dispersed by high power sonic energy into quasi-stable crystalline clusters ranging from 25 to 120 nm. The resulting particles, referred to herein as direct-coated particles, exhibit a significantly larger magnetic moment than colloidal particles of the same overall size, such as those described by Molday or Owen et al.

There exists a tremendous need for advancing the limits of detection, increasing resolution, obtaining information at a molecular level, detecting diseases in their early stages, and obtaining physiological information through MRI investigation. These challenges require an improvement in contrast agent sensitivity, selectivity, blood-circulation time and also characterization of biomarkers and targeting ligands.

As a result of the foregoing, a method and/or composition by/with which nanoparticles would provide enhanced relaxivity, signal-to-noise ratio and targeting abilities with resistance to agglomeration and an ability to control particle size, blood clearance rate and distribution would be extremely useful.

### BRIEF DESCRIPTION OF THE INVENTION

The present invention provides methods and compositions for improved medical diagnostic imaging. A novel contrasting agent is disclosed for use in MRI. The agent consists of conjugated monoclonal antibodies (mAb) directed against the murine isoform of an endothelial cell activation marker, such as, but not limited to, the murine isoform of anti-ICAM (CD54 endothelial cell activation marker). Typically, targeted MRI contrast agents provide enhanced relaxivity, improved signal-to-noise, targeting ability, and resistance to agglomeration. Methods of making such MRI contrast agents afford better control over particle size, and methods of using such MRI contrast agents typically afford enhanced blood clearance rates and distribution. CD54-FF is used as an MRI contrast agent in targeting vascular endothelial cells comprising a BSA coated iron oxide particle conjugated to a mono-thiolated anti-CD54. The quenched complex is stored in D1H₂0

The present invention is directed to methods for using targeted contrast agents in an imaging technique such as MRI. Such uses can involve delivery to cells in vitro and/or delivery to a mammalian subject in vivo.

### DESCRIPTION OF FIGURES

FIG. 1: Summary for FF prepared for MRI. BSA coated iron oxide particles were subjected to the series of separation and concentration steps in order to obtain smaller size particles in the right matrix and concentration for the conjugation step. Then, FF was reacted with SMCC and conjugated to the mono-thiolated antibody. Resulting FF-MAb conjugate was quenched and washed and stored in DI H20.

FIG. 2: Targeting of anti-ICAM/FF particles to mouse endothelial cells (fluorescence microscopy)

FIG. 3: Targeting of anti-ICAM/FF particles to mouse endothelial cells (NMR minispec)

FIG. 4: T2 Relaxation after injection at 5 mg/kg FF

FIG. 5: T2 Relaxation after injection at 15 mg/kg FF

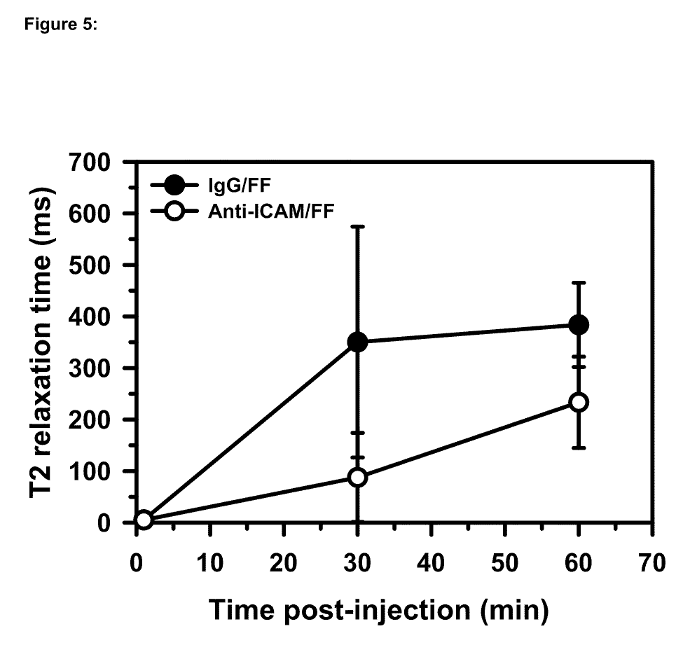

FIG. 6: T2 Relaxation in different organs after 60 min at 5 mg/kg FF

FIG. 7: T2 Relaxation in different organs after 60 min at 15 mg/kg FF

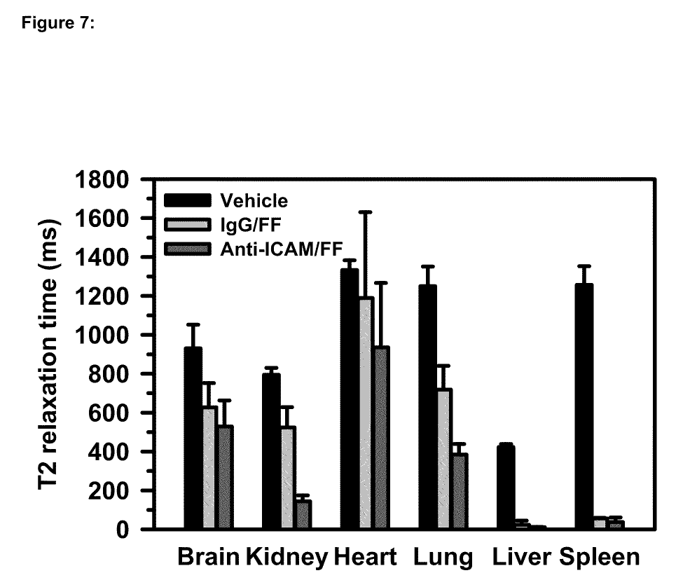

### DETAILED DESCRIPTION OF THE INVENTION

The present invention utilizes a coated, magnetic particle comprising a nanoparticle core of magnetic material, and a base coating material on the magnetic core (U.S. Pat. No. 6,365,362). These magnetic particles are characterized by extremely low non-specific binding. The magnetic core material of the particles described may comprise at least one transition metal oxide and a suitable base coating material comprises a protein. Proteins suitable for coating magnetic particles include but are not limited to bovine serum albumin and casein. The additional coating material may be the original coating proteins or one member of a specific binding pair which is coupled to the base material on the magnetic core. Exemplary specific binding pairs include biotin-streptavidin, antigen-antibody, receptor-hormone, receptor-ligand, agonist-antagonist, lectin-carbohydrate, Protein A-antibody Fc, and avidin-biotin. The member of the specific binding pair may be coupled to the base coating material through a bifunctional linking compound. Exemplary biofunctional linking compounds include succinimidyl-propiono-dithiopyridine (SPDP), and sulfosuccinimidil-4-[maleimidomethyl]cyclohexane-1-carboxylate (SMCC), however a variety of other such heterobifunctional linker compounds are available from Pierce, Rockford, Ill.

The coated magnetic particles of the invention preferably have between 70-90% magnetic mass. A major portion of the magnetic particles have a particle size in the range of 90-150, preferably 15 to 70 nm. Particles may be synthesized such that they are more monodisperse, e.g., in the range of 15 to 30 nm. The particles of the invention are typically suspended in a biologically compatible medium.

Often it is desirable to image activation dysfunction and/or death of the vascular luminal endothelium which occurs in a variety of disease states—cancer, cardiovascular, cerebrovascular, and auto immune disease just to name a few. As a result, the integrity of the endothelium may be compromised resulting in its partial or complete destruction in one or more regions of a vascular bed. The ability to visualize in vivo the location and degree of such damage could provide potentially useful diagnostic and prognostic information. Such information might further aid in the delivery and monitoring of endothelial target specific therapies. Monoclonal antibodies (mAb) functionalized through conjugation to magnetic nanoparticles are used in the present invention as an MRI contrast agent.

Activation dysfunction and/or death of the vascular luminal endothelium occurs in a variety of disease states—cancer, cardiovascular, cerebrovascular, and auto immune disease just to name a few. As a result, the integrity of the endothelium may be compromised resulting in its partial or complete destruction in one or more regions of a vascular bed. The ability to visualize in vivo the location and degree of such damage could provide potentially useful diagnostic and prognostic information. Such information might further aid in the delivery and monitoring of endothelial target specific therapies. The present invention incorporates the use of monoclonal antibodies (mAb) conjugated to magnetic nanoparticles for use as an MRI contrast agent to target an endothelial cell surface activation marker.

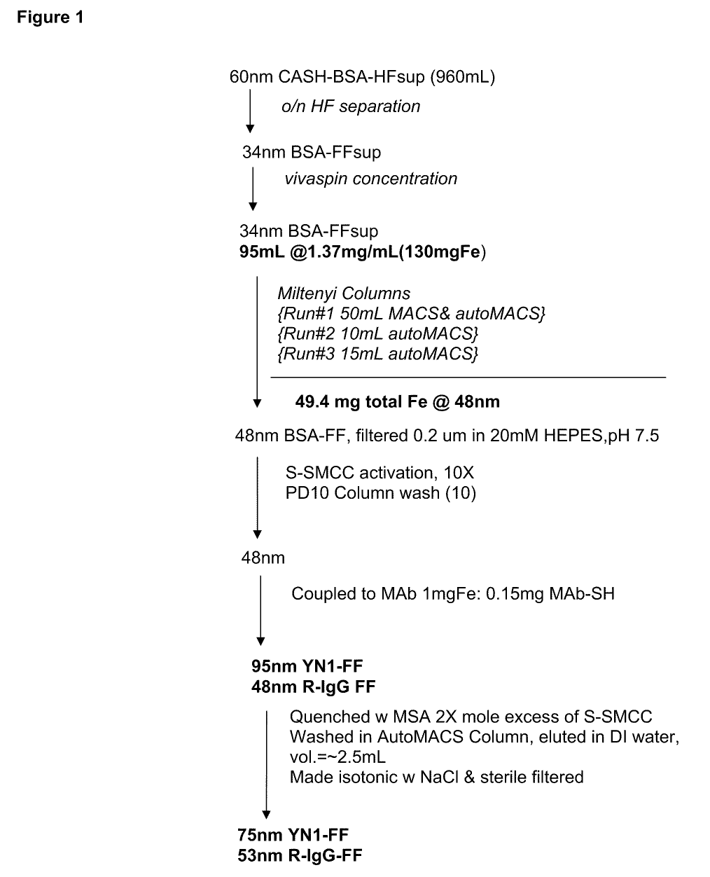

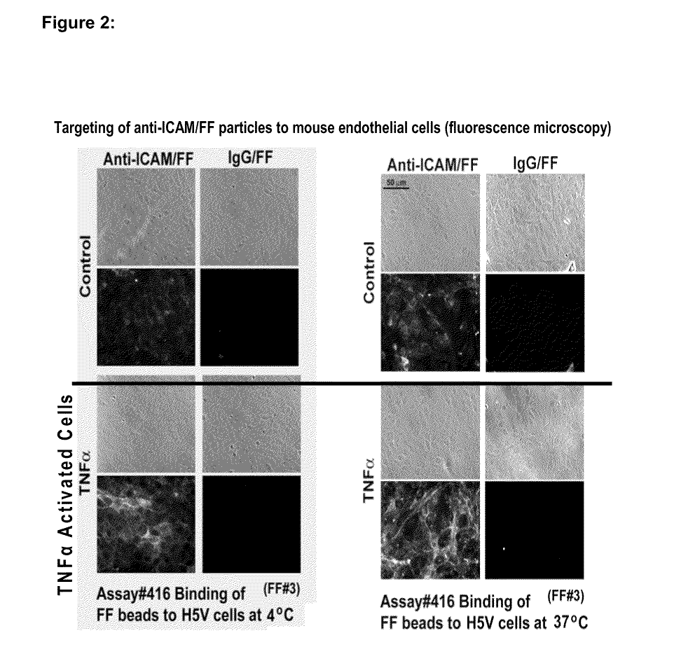

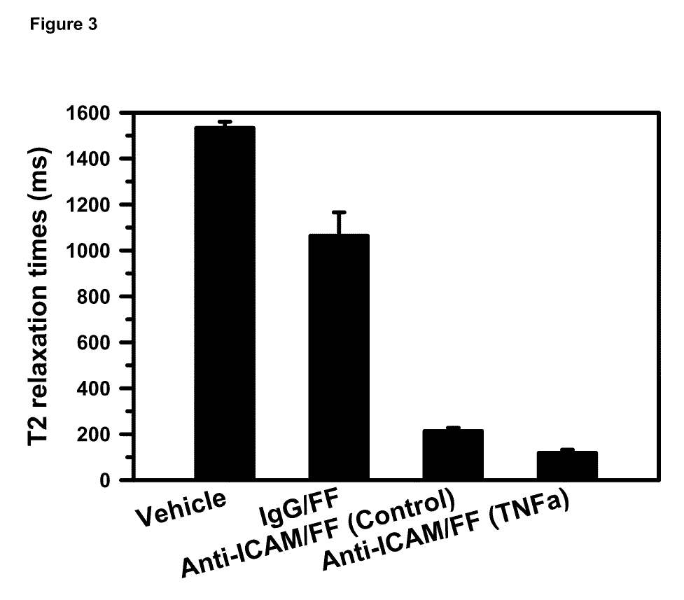

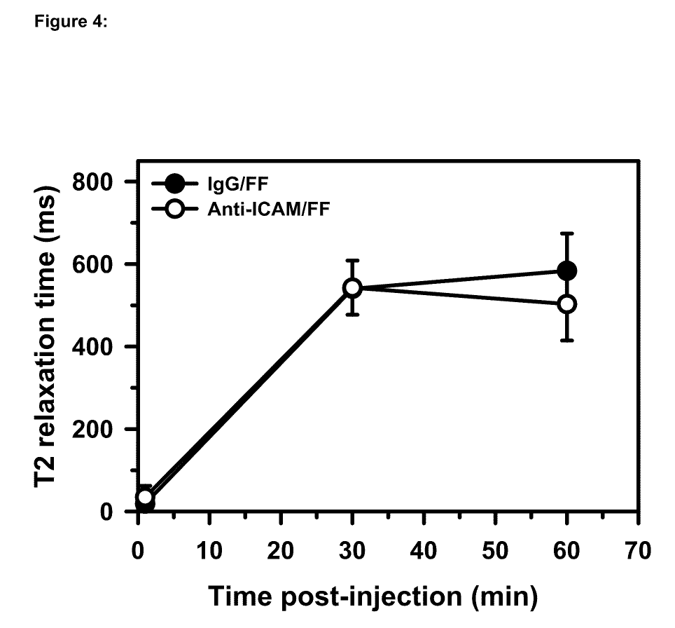

The contrast agent is developed by conjugating rat mAb (clone YN1) directed against the murine isoform of anti-ICAM (CD54 an endothelial cell activation marker) to magnetic ferrofluid (FF) nannoparticles—resulting particle ˜75 nm diameter (FIG. 1). An isotype control is made by conjugating normal rat IgG to FF to produce IgG-FF (64 nm diameter, Fe=11.48 mg/mL). Anti-CD54-FF reactivity in vitro is determined by incubating the agent with murine endothelial cells (EC) treated overnight with TNFα to boost ICAM-1 expression (FIG. 2). After counterstaining with FITC-labeled secondary antibody, cells are inspected by fluorescence microscopy (FM). The cells are then lysed and targeting traced by measuring NMR minispec T2 relaxation times (FIG. 3).: Anesthetized inbred non-reactive mice (N=3) are then injected IV with either 5 mg/kg or 15 mg/kg anti-CD54-FF or IgG-FF and blood collected at 1 min, 30 min and 60 min post-injection (FIGS. 4 & 5). The animals are sacrificed at 1 hr and organs harvested and analyzed by FM and NMR minispec. Lastly, 5 mg/kg is injected IV into 4 mice 2 were pre-treated with TNFα, 2 without. 4 other mice (2 TNFα+, 2 TNFα−) received 5 mg/kg IgG-FF and one control received no IV infusion. After 1 hour the animals are sacrificed and stored at 4 C. All 9 cadavers are then imaged using a 7 T 21 cm Varian MRI instrument for small animals with a 108/38 mm (O.D./I.D.) quadrature birdcage imaging RF coil. T2 and T2* images of chest and abdomen are performed. Duration of imaging is 1 hour/animal, with 30 min/animal for data analysis. Changes in T2 and T2* are calculated to determine specific targeting.

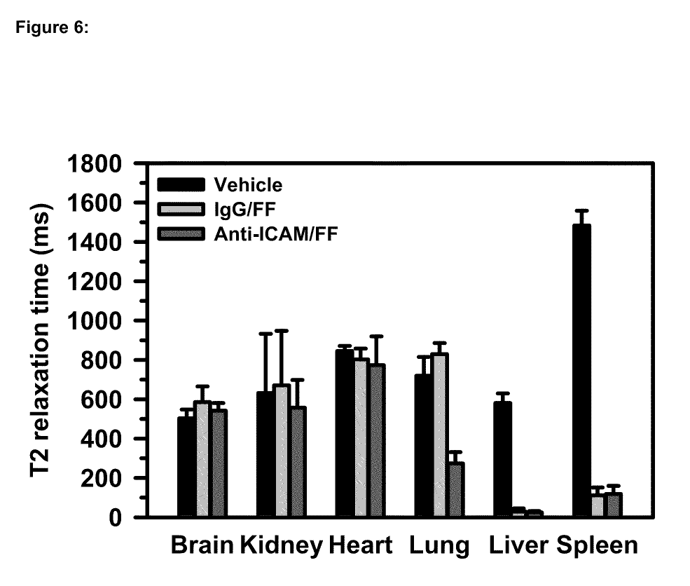

Anti-CD54-FF fluorescence tracing (2^{nd} mAb staining) and T2 relaxation times shows specific targeting to cultured mouse endothelial cells vs. control IgG/FF, both at 4 C or 37 C with the signal higher at 37 C (FIG. 2). Mice injected IV (n=3) with anti-ICAM/FF vs. IgG/FF either at 15 mg/kg or 5 mg/kg, showed substantial CD54-FF targeting of the liver and spleen with somewhat less in kidney and lung. Heart and brain also showed measurable concentrations of the contrast agent. Of the next nine mice imaged, the IgG-FF control enhancement is localized to the spleen and liver only in TNFα+/−animals, while CD54-FF injected animals showed decreased T2 relaxation times in the organs of the TNFα+ animals vs. the TNFα negative group (FIGS. 6 & 7).

CD54-FF functions as an MRI contrast agent targeting activated vascular endothelial cells in multiple organs including the brain as demonstrated by the decreased relaxation times in animals pre-treated with TNFα cytokine. While the data suggests that the most specific targeting is to the lung, the spleen and liver showed increased concentrations for both IgG and CD54-FF, most likely due to Fc-mediated uptake by the reticulo-endothelial system. In addition, 5 C vs. 37 C data from the cultured cell line studies also indicates that these nanoparticles may be endocytosed by endothelial cells.

While embodiments of present invention have been described and specifically exemplified above, it is not intended that the invention be limited to such embodiments. Various modification may be made thereto without departing from the spirit of the present invention, the full scope of the improvements are delineated in the following claims.

I claim:

1. A method for imaging comprising:
a. obtaining a subject for in vivo imaging;
b. introducing a contrast agent into said subject wherein said contrast agent substantially comprises an endothelial cell activation marker coupled to a superparamagnetic nanoparticle having a biofunctional polymer base coating;
c. allowing said contrast agent to interact with the vascular lumen;
d. imaging said interaction wherein said imaging is MRI; and
e. analyzing said imaging for specific target areas.

2. The method of claim 1 wherein said endothelial cell marker is anti-ICAM.

3. The method of claim 1 wherein said contrast agent is anti-CD54-FF.

4. The method of claim 1 wherein said paramagnetic nanoparticle has at least one transition metal oxide in its core.

5. The method of claim 1 wherein said base coating is from a group consisting of protein, bovine serum albumin, casein and combinations thereof.

6. A targeted MRI contrast agent used for in vivo imaging comprising:
a. a colloidal nanoparticle core having at least one transition metal oxide;
b. said nanoparticle having a biofunctional polymer base coating wherein said polymer is from a group consisting of protein, bovine serum albumin, casein, and combinations thereof; and
c. a monoclonal antibody functionalized through said nanoparticle.

7. The contrast agent of claim 2 wherein said monoclonal antibody is anti-CD 54.

8. The contrast agent of claim 2 wherein said nanoparticle is less than 75 nm diameter.
